(19) United States
(12) Patent Application Publication
(10) Pub. No.: US 2003/0049260 A1
(43) Pub. Date: Mar. 13, 2003

(54) Method of improving cognitive function

(76) Inventor: Leonard Bell, (US)

Correspondence Address: Mark Farber, Alexion Pharmaceuticals Inc., 352 Knotter Drive, Cheshire, CT, 06410, US

(21) Appl. No.: 10/191,238

(22) Filed: Jul. 1, 2002

Related U.S. Application Data
(60) Provisional application No. 60/307,968, filed on Jul. 26, 2001.

(51) Int. Cl. A61K 39/395; A61K 31/00

(52) U.S. Cl. 424/146.1; 514/1

(57) ABSTRACT

Methods are useful for determining the effectiveness of anti-inflammatory compounds in reducing the proportion of patients exhibiting an accelerated decline in cognitive function. Reductions in decline of cognitive function in patients are provided by administering an anti-inflammatory compound.

### BACKGROUND

1. Technical Field

This disclosure relates to methods of limiting decline in cognitive function in a subject within a susceptible patient population by administering an anti-inflammatory compound to the subject. Methods for determining the effectiveness of an anti-inflammatory compound in limiting decline of cognitive function is also described.

2. Background of Related Art

Coronary artery disease is often characterized by lesions or occlusions in the coronary arteries which may result in inadequate blood flow to the myocardium, or myocardial ischemia, which is typically responsible for such complications as angina pectoris, necrosis of cardiac tissue (myocardial infarction), and sudden death. In some cases, coronary artery disease may be treated by the use of drugs and by modifications in behavior and diet. In other cases, dilatation of coronary arteries may be achieved by such procedures as angioplasty, laser ablation, atherectomy, catheterization, and intravascular stents.

For certain patients, coronary artery bypass grafting (CABG) is the preferred form of treatment to relieve symptoms and often increase life expectancy. CABG consists of direct anastomosis of a vessel segment to one or more of the coronary arteries. For example, a reversed segment of the saphenous vein may be grafted at one end of the ascending aorta as an arterial blood source and at the other end to a coronary artery at a point beyond the arterial occlusion. Alternatively, the internal mammary artery is located in the thoracic cavity adjacent the sternum and is likewise suitable for grafting to a coronary artery, such as the left anterior descending artery.

During the CABG surgery, the heart may be stopped from beating, to facilitate the anastomosis procedures. While the heart is not beating, extracorporeal circulation of the blood supports most of the patient's body (excluding the heart and, to some extent, the lungs). A cardiopulmonary bypass (CPB) machine receives deoxygenated blood from the patient's body, adds oxygen to the blood, and pumps the oxygenated blood back into the patient's body.

Although CABG surgery has substantially improved the therapeutic outcome of patients with advanced myocardial ischemia, the recovery period may be often traumatic to the patient with significant attendant risks.

Cognitive decline, or “pump-head” as it is frequently referred to in cardiac surgical settings, is a frequent complication of cardiopulmonary bypass surgery. Its etiology is likely multi-factorial, including periprocedural showers of microemboli, perfusion pressure, systemic inflammation, and is accentuated by the presence of demographic risk factors including age, education, and genetic predisposition (Ann Thorac Surg 1995;59:1326-30). Inflammation occurs in the cerebral microvasculature and surrounding neural tissue leading to patchy areas of necrosis and cerebral swelling. It is estimated that measurable cognitive decline occurs in approximately 50-80% of patients at hospital discharge falling to a frequency of approximately 10-30% at six months postop (N Engl J Med 2001; 344:395-402). It has recently been shown that cognitive decline persists in a substantial proportion of patients, with an incidence of approximately 42% at five years, and that the extent of decline at hospital discharge is a good predictor of the cognitive function five years later (N Engl J Med 2001;344:395-402). Hence, particularly as the population undergoing bypass continues to grow older, interventions to limit postoperative cognitive decline may have far-reaching effects on subsequent patient function. Even in patients undergoing CABG surgery without concomitant cardiopulmonary bypass, emboli from manipulation of the heart or the proximal aortic anastomosis may result in cerebral embolian inflammation with subsequent cognitive dysfunction.

Multiple tests have been employed to measure changes in cognitive function in acute and chronic settings. Typically, these tests are selected so as to measure specific domains of cognitive function. These measurements in an individual or group are then compared to measurements obtained in a reference group so as to determine whether the studied individual or group may be considered within the limits of the reference group. The use of cognitive tests to measure serial changes in performance of an individual or group is somewhat less common. Nonetheless, changes in individual tests in subjects have been suggested to indicate clinically important changes in cognitive function in a variety of settings.

One of the most sensitive, and easily applied, tests of cognitive function is the Symbol Digit Modalities Test (SDMT). SDMT is a validated, standardized test which involves the conversion of meaningless geometric designs into written number responses; scores are generated during a 90 second testing period with a range of 0-90. In this manner, subject scores may be compared against a separate population or against a test performed on a different occasion by the same subject. Since the test requires the accurate and speedy translation of nonverbal symbols into numbers, the test measures a wide array of integrated cognitive functions in both the left and right hemispheres. While the left hemisphere is widely believed to play substantial roles in processing language symbols, speaking, writing, and reading, the right hemisphere is believed to be dominant with respect to visual-perceptual, spatial-constructional, and non-verbal reasoning. With these functions generally separated between right and left hemispheres, the process of substituting written numbers for geometric designs requires efficient operations of disparate locations in each hemisphere as well as efficient communication between the hemispheres via the forebrain commissures. The sensitivity of SDMT derives from its measurement of physically disparate yet integrated cognitive functions requiring bilateral processing in an efficient manner. Hence, a “brain-damaged” subject may have normal or above normal IQ and not exhibit any apparent defects in visual, manual motor, or speech functions, but show reduced efficiency in processing the steps required for execution of SDMT. Indeed, the sensitivity of SDMT is highlighted by the observation that, “SDMT scores have shown the most frequent and marked impairment of all the comprehensive neuropsychological test batteries used.” (Symbol Digit Modalities Test Manual, p.1) Clinically meaningful changes in SDMT have been observed in association with anatomic brain changes, with aging, and during drug testing.

Changes in SDMT are a sensitive indicator of the observed decline in cognitive function found in association with normal aging. It has been widely observed that there is a greater decline in function at older ages. Generally, a 7-10% decline in cognitive function has been observed over 6-10 years. For monozygotic or dizygotic twins, a significant 8% decline was observed during a 5 year period starting from a baseline age of 56 years old (Arch Neurol 1992;49:476-81). In the Atherosclerosis Risk in Communities study which included 4,870 subjects aged 58-70 years old, there was an average 7% decline over 6 years (Neurology 2001;56:42-48). In a study of normotensive subjects followed over a 10 year period with baseline age ranging from 59-77 years old (average 72 years old) to an end of study range of 69-87 years old, symbol digit score decline by 3.94 points (Neurology 1998;51:986-993). Additionally, it was observed that even in younger subjects, i.e., less than 58 years of age, the concomitant presence of cardiovascular risk factors gene&ally, and diabetes or hypertension in particular, was associated with an increased rate of decline in symbol digit of approximately 0.9 and 0.4 points, respectively, over a six year period as compared to younger individuals without these risk factors (Neurology 2001;56:42-48). It was further suggested that one of the benefits of treatment of concomitant cardiovascular risk factors in this younger population might be an attenuation of the significant decline in cognitive function over such a relatively brief period of time.

Numerous studies have further noted that there is an expected “learning effect” that occurs with repeat administration of the SDMT, as well as with other standardized cognitive tests. Empirical observations show that re-testing alone, without any intervention, should actually be associated with an increase in SDMT score of approximately 6-7% within a 30 day period (Symbol Digit Modalities Test Manual, p.9). Hence, it is apparent that any decline in scores with subsequent re-testing likely underestimates the true decline in function due to the attenuating effect of concomitant learning.

The sensitivity of SDMT for identifying subtle anatomic brain lesions was confirmed in a large MRI population study. In a study of 3,397 subjects without stroke, it was found that 28% of these subjects had MRI lesions (Stroke 1997;28:1158-1164). Additionally, it was observed that symbol digit testing showed a statistically significant 10% reduction from a mean score of 41.0 in subjects without anatomic lesions to a mean score of 36.9 in subjects with such structural brain lesions.

As shown in the following table, several cardiac and psychotropic drugs have been observed to significantly influence SDMT results in acute and chronic settings. An approximately 10% change from baseline is found to be clinically meaningful.

| | Change in | | |
|---|---|---|---|
| Drug | Symbol Digit | Duration | Reference |
| Methamphetamine | Significant 14% | 5-6 years | Am J Addict |
| | reduction in | of heavy | 2000;9:222-231 |
| | drug group as | use | |
| | compared to a | | |
| | control group | | |
| Triazolam | Significant | 1-2 | NEJM |
| benzodiazepine | 10-20% reduction | hours | 1991,324:1691-8 |
| Atenolol vs. | Significant | 12 | Ann Int Med |
| Nifedipine | 10-12% reduction | weeks | 1992;116:615-623 |
| | w/nifedipine as | | |
| | compared to | | |
| | atenolol | | |
| Alfentanil and | Alfentanil- | 20 | Anesth. Analg |
| Ketamine | 10-12% decline | minutes | 1998;86:1250-6 |
| | Ketamine- | | |
| | 5-10% decline | | |
| Sertraline vs. | Sertraline-max. | 12 | An J Geriatr |
| Fluoxetine SSRI's | 21% increase | weeks | Psychiatry |
| | Fluoxetine-mas. | | 1999;7:221-227 |
| | 10% increase | | |
| Propranolol | 10-15% decline | 8 | Br. J. Clin Pharmac |
| | as compared to | hours | 1984;17:31-36 |
| | placebo | | |

The sensitivity of tests which include measures of psychomotor speed, such as the symbol digit test, as opposed to tests of memory, to identify declines in cognitive function in the CABG setting has been illustrated in a previous study of 155 patients undergoing CABG (Circ 1994;90(part 2):II-250-11-255). In this earlier study, only tests that included a measure of psychomotor speed—digit symbol, Trails B, and Pegs—showed significant declines postoperatively, while tests that measured primarily memory—Buschke total recall test, Buschke consistent long-term retrieval test, and Wechsler memory scale—did not show declines in score.

Significant changes in cognitive function are regularly observed in a large proportion of patients who undergo bypass surgery. It has recently been shown that these acute changes in function found at hospital discharge persist for at least five years. Additionally, a highly sensitive test incorporating an assessment of the function of disparate brain regions, the symbol digit modalities test, has been used to identify clinically significant changes in cognitive function with multiple cardiovascular and psychotropic drug interventions and during the aging process. Employment of this test shows that nearly one half of bypass patients have a 10% decline in function from the brief period of time extending from pre-bypass surgery to hospital discharge and that this finding represents a substantial acceleration in the cognitive decline normally expected in the elderly population, by analogy to other studies with patients of similar age (see Neurology 1998, 51:986-993; Neurology 2001, 56:42-48). Finally, a recent analysis suggests that hospital discharge symbol digit function is a good predictor of overall cognitive function five years following hospital discharge. It would be desirable to develop a method to test the efficacy of a therapeutic candidate to reduce the proportion of patients suffering an accelerated 10% decline in symbol digit score at hospital discharge.

It would also be advantageous to provide a method of limiting a decline of cognitive function in individuals subject to an event leading to neurocognitive damage, such as, for example, in patients having undergone CABG involving CPB.

### SUMMARY

A method of limiting decline in cognitive function in a patient by an effective cognitive function enhancing amount of an anti-inflammatory compound has now surprisingly been found. In another aspect, a method for determining the effectiveness of an anti-inflammatory compound in limiting decline or improving cognitive function has been discovered. This method includes administering an anti-inflammatory compound to a subject group including at least one in a susceptible patient population and comparing the cognitive function in the subject group to cognitive function in a control sample of patients. A decrease in the proportion of patients exhibiting an accelerated decline in symbol digit score in the subject group indicates effectiveness of the compound.

### DETAILED DESCRIPTION OF PREFERRED EMBODIMENTS

The methods of limiting decline in cognitive function in accordance with this disclosure include the step of administering an effective cognitive function enhancing amount of an anti-inflammatory compound to a subject within a susceptible patient population. A susceptible patient population is a group of individuals likely to experience an accelerated decline in cognitive function (compared to the decline naturally associated with aging). Such groups include, but are not limited to: patients having chronic neurological diseases (such as, for example, Alzheimer's disease, Parkinson's disease, etc.); patients having severe hypertension; patients having acute neurological disease (such as, for example, cerebral trauma, stroke victims, etc.); and patients undergoing a procedure which involves cardiopulmonary bypass (such as, for example, CABG or heart transplant) or cerebrovascular surgery (such as, for example, carotid endarterectomy) or off-pump cardiac surgery.

An effective cognitive function enhancing amount of an anti-inflammatory compound is an amount sufficient to reduce the decline in cognitive function of a patient compared to the cognitive decline in a patient not receiving the compound. The specific amount which constitutes an effective cognitive function enhancing amount depends on the specific anti-inflammatory compound employed.

Anti-inflammatory compounds which can be administered in accordance with the methods described herein include non-steroidal anti-inflammatory actives or drugs (NSAIDS). The NSAIDS can be selected from the following categories: propionic acid derivatives; acetic acid derivatives; fenamic acid derivatives; biphenylcarboxylic acid derivatives; and oxicams. All of these NSAIDS are fully described in the U.S. Pat. No. 4,985,459 to Sunshine et al., issued Jan. 15, 1991, incorporated by reference herein. Most preferred are the propionic NSAIDS including, but not limited to aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Another useful class of anti-inflammatory compounds include inhibitors of cyclooxygenase-1 (COX-1) and inhibitors of cyclooxygenase-2 (COX-2). Also useful are the steroidal anti-inflammatory drugs including hydrocortisone and the like. Particularly useful are anti-inflammatory compounds which reduce neutrophil activation or monocyte activation by greater than about 30%.

Preferred anti-inflammatory compounds are compounds which bind to or otherwise block the generation and/or activity of complement components. A specific class of such compounds which are particularly useful are antibodies specific to a human complement component.

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, In The Merck Manual, 16^{th} Edition.

The complement cascade progresses via the classical pathway or the alternative pathway. These pathways share many components, and while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease (which is different in each pathway) to yield C3a and C3b. Other pathways activating complement attack can act later in the sequence of events leading to various aspects of complement function.

C3a is an anaphylatoxin (see discussion below). C3b binds to bacterial and other cells, as well as to certain viruses and immune complexes, and tags them for removal from the circulation. (C3b in this role is known as opsonin.) The opsonic function of C3b is generally considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone (Fearon, in Intensive Review of Internal Medicine, 2^{nd} Ed. Fanta and Minaker, eds. Brigham and Women's and Beth Israel Hospitals, 1983).

C3b also forms a complex with other components unique to each pathway to form classical or alternative C5 convertase, which cleaves C5 into C5a and C5b. C3 is thus regarded as the central protein in the complement reaction sequence since it is essential to both the alternative and classical pathways (Wurzner, et al., Complement Inflamm. 8:328-340, 1991). This property of C3b is regulated by the serum protease Factor I, which acts on C3b to produce iC3b. While still functional as opsonin, iC3b cannot form an active C5 convertase.

C5a is another anaphylatoxin (see discussion below). C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

As mentioned above, C3a and C5a are anaphylatoxins. These activated complement components can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

Any compounds which bind to or otherwise block the generation and/or activity of any of the human complement components, such as, for example, antibodies specific to a human complement component are useful herein. Some compounds include 1) antibodies directed against complement components C-1, C-2, C-3, C-4, C-5, C-6, C-7, C-8, C-9, Factor D, Factor B, Factor P, MBL, MASP-1, AND MASP-2 and 2) naturally occurring or soluble forms of complement inhibitory compounds such as CR1, LEX-CR1, MCP, DAF, CD59, Factor H, cobra venom factor, FUT-175, y bind protein, complestatin, and K76 COOH. Suitable compounds for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. Particularly useful complement inhibitors are compounds which reduce the generation of C5a and/or C5b-9 by greater than about 30%. A particularly useful anti-C5 antibody is h5G1.1-scFv. Methods for the preparation of h5G1.1-scFv are described in U.S. patent application No. 08/487,283 filed Jun. 7, 1995 now U.S. Pat. No. ______ and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by this reference. h5G1.1-scFv is currently undergoing clinical trials under the tradename Pexelizumab.

Any known test can be used to test the cognitive function of the patient. A particularly useful test is the Symbol Digit Modalities Test (SDMT) described above. The SDMT is described in detail in a manual published by Western Psychological Services, Los Angeles, Calif. (Seventh Printing, February 1995) the contents of which are incorporated herein by this reference.

The following non-limiting example is included to illustrate the present invention but is not intended to limit the scope thereof.

### EXAMPLE

### Assessment of Percent Change in Symbol Digit Scores at Discharge Post CABG:

### Clinical Relevance of a 10% or 15% Decline in Symbol Digit Score

A randomized, multi-center, double-blind, placebo-controlled study was conducted of a humanized single chain antibody that binds to C5, Pexelizumab, administered to patients who underwent CPB with CABG.

The study population consisted of individuals who elected to undergo non-emergent coronary-artery bypass graft (CABG) surgery, with or without valve surgery, which required the use of a cardiopulmonary bypass (CPB) machine. There were 692 patients involved in the study of bypass related changes in cognitive function as measured by the Symbol Digit Modalities Test (SDMT). Of these, 617 patients underwent CABG only.

A total of 399 CABG only patients received one of the following treatment combinations: i) Bolus 2.0 mg/kg Pexelizumab followed by 0.05 mg/kg/hr Pexelizumab for 24 hours; or ii) placebo. The Pexelizumab or matching placebo was provided as a solution for injection in 30 ml vials with a concentration of 2 mg/ml. Patients received the bolus of study medication ten (10) minutes before the initiation of cardiopulmonary bypass via a unique line. The drug was not to be combined with other medication given via this route. The infusion began immediately following bolus administration, and continued for 24 hours at a constant drip rate.

Patients were evaluated at an initial screening visit at which the SDMT was administered to establish a baseline result for each patient.

The results of the study for day 4 and day 30 for patients who underwent CABG with or without valve surgery are presented in Tables I and II, respectively, below. The relevance of changes in symbol digit scores at day 4 and day 30 was studied.

TABLE I
All procedures - All Patients

| | | Placebo | Bolus/Infusion | Chi-Square |
|---|---|---|---|---|
| % Change | Time Point | (n = 224) | (n = 221) | P value |
| −20% | 4 d | 40.4 | 30.3 | .027 |
| −15% | 4 d | 47.1 | 34.0 | .005 |
| −10% | 4 d | 55.2 | 41.2 | .003 |
| Decline | 4 d | 70.7 | 62.9 | .080 |
| Improve | 4 d | 29.3 | 37.1 | .080 |
| +10% | 4 d | 13.9 | 20.8 | .054 |
| +15% | 4 d | 12.1 | 16.3 | .207 |
| +20% | 4 d | 9.4 | 13.1 | NS |
| +/−10% | 4 d | | | .010 |
| +/−15% | 4 d | | | .018 |
| +/−20% | 4 d | | | .069 |

TABLE II
All procedures - All Patients

| | | | | Chi-Square |
|---|---|---|---|---|
| % Change | Time Point | Placebo | Bolus/Infusion | P value |
| −20% | 30 d | 9.8 | 8.1 | NS |
| −15% | 30 d | 12.6 | 10.2 | NS |
| −10% | 30 d | 22.9 | 12.7 | .007 |
| Decline | 30 d | 32.4 | 19.8 | .004 |
| Improve | 30 d | 67.6 | 80.2 | .004 |
| +10% | 30 d | 41.1 | 54.8 | .005 |
| +15% | 30 d | 31.3 | 41.6 | .03 |
| +20% | 30 d | 22.9 | 31.0 | .065 |
| +/−10% | 30 d | | | .006 |
| +/−15% | 30 d | | | .092 |
| +/−20% | 30 d | | | .176 |
| +/−10% | 30 d | | | .006 |
| +/−15% | 30 d | | | .092 |
| +/−20% | 30 d | | | .176 |

In patients undergoing CABG only, it was observed that 56% of placebo patients compared to 40% of Pexelizumab bolus/infusion patients obtained a decline in symbol digit score of greater than or equal to 10% at Day 4 (p=0.02 for placebo vs. active).

From the current data, it appears that a large proportion of post-CABG patients acutely suffer a markedly accelerated cognitive decline. The cognitive decline measured over a period of

What is claimed is:

1. A method of reducing the extent of cognitive decline in a subject comprising:
administering to a subject susceptible to accelerated cognitive decline an effective cognitive function enhancing amount of an anti-inflammatory compound.

2. The method of claim 1, wherein the subject is selected from the group consisting of patients having chronic neurological diseases, patients having severe hypertension, patients have acute neurological diseases, patients undergoing a procedure which involves cardiopulmonary bypass, patients undergoing cerebrovascular surgery and patients undergoing off-pump cardiac surgery.

3. The method of claim 2, wherein the subject is a patient undergoing a procedure involving cardiopulmonary bypass.

4. The method of claim 3, wherein the subject is a patient undergoing coronary artery bypass grafting, with or without valve surgery.

5. The method of claim 2, wherein the subject is a patient undergoing off-pump cardiac surgery.

6. The method of claim 1, wherein the anti-inflammatory compound is a complement inhibitor.

7. The method of claim 6, wherein the complement inhibitor is selected from the group consisting of a) antibodies directed against complement components C-1, C-2, C-3, C-4, C-5, C-6, C-7, C-8, C-9, Factor D, Factor B, Factor P, MBL, MASP-1, or MASP-2; and b) naturally occurring or soluble forms of CR1, LEX-CR1, MCP, DAF, CD59, Factor H, cobra venom factor, FUT-1 75, y bind protein, complestatin, or K76COOH 2.

8. The method of claim 7, wherein the antibody directly or indirectly reduces the conversion of complement component C5 into complement components C5a and C5b.

9. The method of claim 8, wherein the anti-C5 antibody is an antibody comprising at least one antibody-antigen binding site, said antibody exhibiting specific binding to human complement component C5, said specific binding being targeted to the alpha chain of human complement component C5, wherein the antibody 1) inhibits complement activation in a human body fluid; 2) inhibits the binding of purified human complement component C5 to either human complement component C3 or human complement component C4; and 3) does not specifically bind to the human complement activation product for C5a.

10. The method of claim 6, wherein the complement inhibitor specifically binds to a component forming the C5b-9 complex.

11. The method of determining effectiveness of an anti-inflammatory compound in reducing the incidence of decline in cognitive function comprising:
administering the compound to a subject group including at least one patient susceptible to an accelerated decline in cognitive function; and
comparing the cognitive function as determined by symbol digit score in the subject group to the cognitive function in a control sample of patients;
wherein a decrease in the proportion of patients exhibiting an accelerated 10% decline in symbol digit score in the subject group indicates effectiveness of the compound.

12. The method of claim 11, wherein the subject group is a group of patients selected from the group consisting of patients having chronic neurological diseases, patients having severe hypertension, patients having acute neurological diseases, patients undergoing a procedure which involves cardiopulmonary bypass, patients undergoing cerebrovascular surgery, and patients undergoing off-pump cardiac surgery.

13. The method of claim 11, wherein the subject group is a group of patients undergoing a procedure involving cardiopulmonary bypass.

14. The method of claim 13, wherein the subject group is a group of patients undergoing coronary artery bypass grafting, with or without valve surgery.

15. The method of claim 11, wherein the subject group is a group of patients undergoing off-pump cardiac surgery.

16. The method of claim 11, wherein the anti-inflammatory compound is a complement inhibitor.

17. The method of claim 16, wherein the complement inhibitor is selected from the group consisting of a) antibodies directed against complement components C-1, C-2, C-3, C-4, C-5, C-6, C-7, C-8, C-9, Factor D, Factor B, Factor P, MBL, MASP-1, or MASP-2; and b) naturally occurring or soluble forms of CR1, LEX-CR1, MCP, DAF, CD59, Factor H, cobra venom factor, FUT-175, y bind protein, complestatin and K76 COOH.

18. The method of claim 16, wherein the antibody directly or indirectly reduces the conversion of complement component C5 into complement components C5a and C5b.

19. The method of claim 18, wherein the anti-C5 antibody is an antibody comprising at least one antibody-antigen binding site, said antibody exhibiting specific binding to human complement component C5, said specific binding being targeted to the alpha chain of human complement component C5, wherein the antibody 1) inhibits complement activation in a human body fluid; 2) inhibits the binding of purified human complement component C5 to either human complement component C3 or human complement component C4; and 3) does not specifically bind to the human complement activation product for C5a.

20. The method of claim 16, wherein the complement inhibitor specifically binds to a component forming the C5b-9 complex.
